(19) United States
(12) Patent Application Publication
(10) Pub. No.: US 2006/0121111 A1
(43) Pub. Date: Jun. 8, 2006

(54) Formulations of substituted benzoxazoles

(75) Inventors: Kai Zhuang, River Edge, NJ (US); Wendy A. Dulin, Tuxedo, NY (US); Marc S. Tesconi, Monroe, NY (US); Mannching Sherry Ku, Thiells, NY (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/290,731

(22) Filed: Nov. 30, 2005

Related U.S. Application Data
(60) Provisional application No. 60/632,466, filed on Dec. 2, 2004.

(51) Int. Cl. A61K 31/428; A61K 31/423; A61K 31/4184; A61K 9/20

(52) U.S. Cl. 424/464; 514/367; 514/375; 514/394

(57) ABSTRACT

The present invention provides solid dosage formulations of benzoxazole-containing ERβ-selective ligands, and processes for their manufacture.

### CROSS-REFERENCE TO RELATED APPLICATION

The present invention claims benefit of priority from provisional U.S. Patent Application Ser. No. 60/632,466 filed Dec. 2, 2004, which is incorporated herein by reference in its entirety.

### FORMULATIONS OF SUBSTITUTED BENZOXAZOLES

1. Field of the Invention

The present invention relates to solid dosage formulations that include ERβ-selective ligands that contain benzoxazole (or benzothiazole or benzoimidazole), and processes for manufacture of said formulations, more particularly to novel formulations and processes for manufacture of formulations containing the ERβ-selective ligand, ERB-041.

2. Background of the Invention

This invention relates to formulations for substituted benzoxazoles (and benzothiazoles and benzodiazoles), which are useful as estrogenic agents.

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Women's Health & Gender Based Medicine 8: 1155-1166 (1999), Monk and Brodaty, Dementia & Geriatric Cognitive Disorders 11: 1-10 (2000), Hum and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles of Molecular Regulation 351-361 (2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed, some compounds behave as estrogen receptor agonists in some tissues and estrogen receptor antagonists in others. These compounds with mixed activity are called selective estrogen receptor modulators (SERMS) and are therapeutically useful agents (e.g., EVISTA®) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist that sterically hinders the protein sequences required for receptor-coregulatory protein interaction [Pike, et al., EMBO 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

The preparation of exemplary ERβ selective ligands, including 2-(3-fluoro-4-hydroxyphenyl)-7-vinyl-1,3-benzoxazol-5-ol (ERB-041), is described in U.S. Pat. No. 6,794,403, incorporated herein by reference in its entirety.

As mentioned above, estrogens affect a panoply of biological processes. In addition, where gender differences have been described (e.g., disease frequencies, responses to challenge, etc.), it is possible that the explanation involves the difference in estrogen levels between males and females.

Given the importance of these compounds as pharmaceutical agents, it can be seen that effective formulations for delivery of the compounds is of great import. This invention is directed to these, as well as other, important ends.

### SUMMARY OF THE INVENTION

In some embodiments, the present invention provides pharmaceutical formulations comprising a pharmaceutically effective amount of an active pharmacological agent and a carrier or excipient system, the carrier or excipient system comprising:

a) a solubilizer/wetting agent component comprising from about 1% to about 60% by weight of the pharmaceutical formulation;

b) an optional co-solubilizer component comprising from about 0.04% to about 15% by weight of the pharmaceutical formulation;

c) a diluent/adsorbent component comprising from about 10% to about 60% by weight of the pharmaceutical formulation;

d) an optional second diluent/adsorbent component comprising from about 10% to about 88% by weight of the pharmaceutical formulation;

e) a disintegrant component comprising from about 0.5% to about 8% by weight of the pharmaceutical formulation;

f) an optional glidant component comprising from about 0.05% to about 5.0% by weight of the pharmaceutical formulation; and

g) an optional lubricant component comprising from about 0.001% to about 10.0% by weight of the pharmaceutical formulation; wherein the active pharmacological agent has the Formula I: 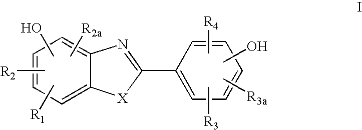
wherein

R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅ and R₆ are each, independently hydrogen, alkyl of 1-6 carbon atoms, or aryl of 6-10 carbon atoms;

X is O, S, or N R₇; and

R₇ is hydrogen, alkyl of 1-6 carbon atoms or aryl of 6-10 carbon atoms, —COR₅, —CO₂R₅ or —SO₂R₅;

or a pharmaceutically acceptable salt thereof.

In some embodiments, X is O. In some further embodiments, R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some embodiments, the active ingredient is 2-(3-fluoro-4-hydroxyphenyl)-7-vinyl-1,3-benzoxazol-5-ol (ERB-041) or a pharmaceutically acceptable salt thereof.

The term halogen refers to chloro, bromo, fluoro or iodo, preferably fluoro. The alkyl of 1-6 carbon atoms (used alone or as part of a group e.g. alkoxy) may be a straight or branched alkyl e.g. methyl, ethyl, n-propyl, i-propyl or n-butyl. The cycloalkyl of 3-8 carbon atoms may be saturated or unsaturated and includes the moieties cyclopropyl, cyclobutyl, cyclopentyl and cyclohexyl. The trifluoroalkyl of 1-6 carbon atoms (used alone or as part of a group) may suitably be trifluoromethyl. Sulfoxoalkyl of 1-6 carbon atoms refers to the group —SO—R wherein R is an alkyl of 1-6 carbon atoms as defined above. Aryl of 6-10 carbon atoms refers to a mono or poly cyclic aromatic group. e.g., phenyl or napthyl. The 5 to 6 membered heterocyclic ring having 1 to 4 heteroatoms selected from O, N or S is a saturated, partially unsaturated or aromatic ring, e.g., a furanyl, pyranyl, pyridinyl, pyrimidinyl, pyrazinyl, morpholinyl, thiomorpholinyl, imidazolyl, oxazolyl, thioxazolyl, thienyl or piperidinyl ring. The alkynyl of 2-7 carbon atoms is a group having at least one triple bond, e.g., ethynyl. The alkenyl of 2-7 carbon atoms is a group having at least one double bond, e.g., vinyl. When the alkyl or alkenyl moieties are substituted they may be substituted with 1 or more substituents as defined above, e.g. by 1, 2 or 3 substituents which may be the same or different.

Preferably, the active pharmacological agent comprises up to about 59% by weight of the pharmaceutical formulation.

In some embodiments, the active pharmacological agent comprises from about 0.5% to about 50% by weight of the pharmaceutical formulation; the solubilizer/wetting agent component comprises from about 20% to about 50% by weight of the pharmaceutical formulation; the optional co-solubilizer component, when present, comprises from about 0.1% to about 5% by weight of the pharmaceutical formulation; the diluent/adsorbent component comprises from about 15% to about 30% by weight of the pharmaceutical formulation; the optional second diluent/adsorbent component, when present, comprises from about 15% to about 30% by weight of the pharmaceutical formulation; the disintegrant component comprises from about 1% to about 6% by weight of the pharmaceutical formulation; the optional glidant component, when present, comprises from about 0.1% to about 1.0% by weight of the pharmaceutical formulation; and the optional lubricant component, when present, comprises from about 0.005% to about 9% by weight of the pharmaceutical formulation.

In some further embodiments, the active pharmacological agent comprises from about 5.0% to about 50% by weight of the pharmaceutical formulation; the solubilizer/wetting agent component comprises from about 25% to about 35% by weight of the pharmaceutical formulation; the optional co-solubilizer component, when present, comprises from about 0.5% to about 3% by weight of the pharmaceutical formulation; the diluent/adsorbent component comprises from about 18% to about 27% by weight of the pharmaceutical formulation; the optional second diluent/adsorbent component, when present, comprises from about 18% to about 27% by weight of the pharmaceutical formulation; the disintegrant component comprises from about 3% to about 5% by weight of the pharmaceutical formulation; the optional glidant component, when present, comprises from about 0.1% to about 0.4% by weight of the pharmaceutical formulation; and the optional lubricant component, when present, comprises from about 0.01% to about 8% by weight of the pharmaceutical formulation.

In still further embodiments, the active pharmacological agent comprises from about 10% to about 30% by weight of the pharmaceutical formulation; the solubilizer/wetting agent component comprises from about 30% to about 40% by weight of the pharmaceutical formulation; the optional co-solubilizer component, when present, comprises from about 0.5% to about 1.5% by weight of the pharmaceutical formulation; the diluent/adsorbent component comprises from about 20% to about 26% by weight of the pharmaceutical formulation; the optional second diluent/adsorbent component, when present, comprises from about 20% to about 26% by weight of the pharmaceutical formulation; the disintegrant component comprises from about 3% to about 5% by weight of the pharmaceutical formulation; the optional glidant component, when present, comprises from about 0.1% to about 0.4% by weight of the pharmaceutical formulation; and the optional lubricant component, when present, comprises from about 0.01% to about 5% by weight of the pharmaceutical formulation.

In some embodiments, the solubilizer/wetting agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar ester of fatty acid, glyceride of fatty acid, or polyglycolized glyceride; preferably, Poloxamer 188.

In some embodiments, the optional co-solubilizer component, if present, comprises one or more of gelatin, polyvinylpyrrolidone (PVP), hydroxypropylmethylcellulose (HPMC), pregelatinized starch, plain starch, hydroxypropylcellulose (HPC), or carboxymethylcellulose (CMC); preferably, polyvinylpyrrolidone K17 (e.g., Povidone K17).

In some embodiments, the diluent/adsorbent component comprises one or more of carboxymethyl cellulose, carboxyethyl cellulose, hydroxyethyl cellulose, microcrystalline cellulose, starch, calcium phosphate, anhydrous dicalcium phosphate, sodium starch glycolate, magnesium carbonate, metal aluminosilicate, or magnesium aluminometasilicate; preferably, magnesium aluminometasilicate (e.g., Neusilin®).

In some embodiments, the optional second diluent/adsorbent component, if present, comprises one or more of a calcium phosphate, anhydrous dicalcium phosphate, carboxymethyl cellulose, carboxyethyl cellulose, hydroxyethyl cellulose, microcrystalline cellulose, starch, sodium starch glycolate, metal aluminosilicate, or magnesium aluminometasilicate; preferably, anhydrous dicalcium phosphate.

In some embodiments, the disintegrant component comprises one or more of croscarmellose sodium, modified cellulose, pregelatinized starch, sodium starch glycolate, crospovidone, starch, alginic acid, sodium alginate, clay, cellulose floc, ion exchange resin, silica, or effervescent system based on food acid and an alkaline carbonate component; preferably, croscarmellose sodium.

In some embodiments, the optional glidant component, if present, comprises one or more of starch, talc, lactose, stearate, dibasic calcium phosphate, magnesium carbonate, magnesium oxide, calcium silicate, silicon dioxide, or silicon dioxide aerogel; preferably silicon dioxide (e.g., Syloid® 244FP).

In some embodiments, the optional lubricant component, if present, comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica such as Aerosil® 200, or sodium chloride; preferably, magnesium stearate.

In some further embodiments, the solubilizer/wetting agent component comprises Poloxamer 188; the optional co-solubilizer component, when present, comprises Povidone K17; the diluent/adsorbent component comprises magnesium aluminometasilicate (e.g., Neusilin®); the disintegrant component comprises croscarmellose sodium; the optional second diluent/adsorbent component, when present, comprises anhydrous dicalcium phosphate; the optional glidant component, when present, comprises silicon dioxide; and the optional lubricant component, when present, comprises magnesium stearate.

In some embodiments, the formulation contains from about 1 mg to about 125 mg, or about 1 mg to about 3 mg, or about 3 mg to about 7 mg, or about 20 mg to about 30 mg, or about 40 mg to about 60 mg, or about 70 mg to about 80 mg, or about 90 mg to about 110 mg of active pharmacological agent.

The present invention further provides processes for preparing a pharmaceutical formulation comprising a pharmaceutically effective amount of an active pharmacological agent and a carrier or excipient system, the carrier or excipient system comprising:

a) a solubilizer/wetting agent component comprising from about 1% to about 60% by weight of the pharmaceutical formulation;

b) an optional co-solubilizer component comprising from about 0.04% to about 15% by weight of the pharmaceutical formulation;

c) a diluent/adsorbent component comprising from about 10% to about 60% by weight of the pharmaceutical formulation;

d) an optional second diluent/adsorbent component comprising from about 10% to about 88% by weight of the pharmaceutical formulation;

e) a disintegrant component comprising from about 0.5% to about 8% by weight of the pharmaceutical formulation;

f) an optional glidant component comprising from about 0.05% to about 5.0% by weight of the pharmaceutical formulation; and

g) an optional lubricant component comprising from about 0.001% to about 10.0% by weight of the pharmaceutical formulation; the process comprising:

i) blending the diluent/adsorbent component, at least a portion of the second diluent/adsorbent component, when present, and at least a portion of the disintegrant component to form a first mixture;

ii) mixing together the solubilizer/wetting agent component, the co-solubilizer component, if present, and the active pharmacological agent to form a second mixture;

iii) mixing the first and second mixtures to form a third mixture;

iv) blending at least a portion of the disintegrant component and the glidant component, if present, and, if present, at least a portion of the second diluent/adsorbent component with the third mixture to form a fourth mixture;

v) adding the optional lubricant component, if present, to the fourth mixture to form a final blend;

wherein the pharmacological agent is 2-(3-fluoro-4-hydroxyphenyl)-7-vinyl-1,3-benzoxazol-5-ol (ERB-041) or a pharmaceutically acceptable salt thereof.

In some embodiments, the pharmacological active agent is micronised. In some embodiments, step (i) is performed in a heated jacketed bowl.

In some embodiments, the solubilizer/wetting agent component and the co-solubilizer component are separately melted prior to mixing with the active pharmacological agent. In further embodiments, the solubilizer/wetting agent component and the co-solubilizer component are melted together prior to mixing with the active pharmacological agent, preferably at a temperature from about 110° C. to about 130° C., preferably about 120° C.

In some embodiments, the solubilizer/wetting agent component, the co-solubilizer component and the active pharmacological agent are melted at a temperature from about 110° C. to about 130° C., preferably 120° C., preferably for about 30 minutes to about 4 hours, preferably until a substantially clear mixture is attained.

In some embodiments, step (iii) is performed at a temperature from about 90° C. to about 130° C., preferably about 100° C. Typically, the heated granulate is then cooled.

In some embodiments, the processes further comprise encapsulating at least a portion of the final blend.

The present invention also provides products of the processes described herein.

In some embodiments, the pharmaceutical formulations of the invention comprise about 40 mg to about 60 mg ERB-041 micronised; about 90 mg to about 110 mg Poloxamer 188; about 2 mg to about 4 mg Povidone K17; about 55 mg to about 75 mg Neusilin®; about 55 mg to about 75 mg anhydrous dicalcium phosphate; about 8 mg to about 12 mg croscarmellose sodium; about 0.01 mg to about 1 mg Syloid® 244FP; and optionally about 1.0 mg to about 2.0 mg magnesium stearate.

### SUMMARY OF DRAWINGS

FIG. 1: Flow diagram of wet melt process.

### DETAILED DESCRIPTION

In some embodiments, the present invention provides pharmaceutical formulations comprising a pharmaceutically effective amount of an active pharmacological agent and a carrier or excipient system, the carrier or excipient system comprising:

a) a solubilizer/wetting agent component comprising from about 1% to about 60% by weight of the pharmaceutical formulation;

b) an optional co-solubilizer component comprising from about 0.04% to about 15% by weight of the pharmaceutical formulation;

c) a diluent/adsorbent component comprising from about 10% to about 60% by weight of the pharmaceutical formulation;

d) an optional second diluent/adsorbent component comprising from about 10% to about 88% by weight of the pharmaceutical formulation;

e) a disintegrant component comprising from about 0.5% to about 8% by weight of the pharmaceutical formulation;

f) an optional glidant component comprising from about 0.05% to about 5.0% by weight of the pharmaceutical formulation; and

g) an optional lubricant component comprising from about 0.001% to about 10.0% by weight of the pharmaceutical formulation;
wherein the active pharmacological agent has the Formula I: 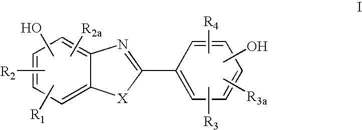
wherein

R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅ and R₆ are each, independently hydrogen, alkyl of 1-6 carbon atoms, or aryl of 6-10 carbon atoms;

X is O, S, or N R₇; and

R₇ is hydrogen, alkyl of 1-6 carbon atoms or aryl of 6-10 carbon atoms, —COR₅, —CO₂R₅ or —SO₂R₅;

or a pharmaceutically acceptable salt thereof.

In some embodiments, X is O. In some further embodiments, R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some embodiments, the active ingredient is 2-(3-fluoro-4-hydroxyphenyl)-7-vinyl-1,3-benzoxazol-5-ol (ERB-041) or a pharmaceutically acceptable salt thereof.

Preferably, the active pharmacological agent comprises up to about 59% by weight of the pharmaceutical formulation.

In some embodiments, the active pharmacological agent comprises from about 0.5% to about 50% by weight of the pharmaceutical formulation; the solubilizer/wetting agent component comprises from about 20% to about 50% by weight of the pharmaceutical formulation; the optional co-solubilizer component, when present, comprises from about 0.1% to about 5% by weight of the pharmaceutical formulation; the diluent/adsorbent component comprises from about 15% to about 30% by weight of the pharmaceutical formulation; the optional second diluent/adsorbent component, when present, comprises from about 15% to about 30% by weight of the pharmaceutical formulation; the disintegrant component comprises from about 1% to about 6% by weight of the pharmaceutical formulation; the optional glidant component, when present, comprises from about 0.1% to about 1.0% by weight of the pharmaceutical formulation; and the optional lubricant component, when present, comprises from about 0.005% to about 9% by weight of the pharmaceutical formulation.

In some further embodiments, the active pharmacological agent comprises from about 5.0% to about 50% by weight of the pharmaceutical formulation; the solubilizer/wetting agent component comprises from about 25% to about 35% by weight of the pharmaceutical formulation; the optional co-solubilizer component, when present, comprises from about 0.5% to about 3% by weight of the pharmaceutical formulation; the diluent/adsorbent component comprises from about 18% to about 27% by weight of the pharmaceutical formulation; the optional second diluent/adsorbent component, when present, comprises from about 18% to about 27% by weight of the pharmaceutical formulation; the disintegrant component comprises from about 3% to about 5% by weight of the pharmaceutical formulation; the optional glidant component, when present, comprises from about 0.1% to about 0.4% by weight of the pharmaceutical formulation; and the optional lubricant component, when present, comprises from about 0.01% to about 8% by weight of the pharmaceutical formulation.

In still further embodiments, the active pharmacological agent comprises from about 10% to about 30% by weight of the pharmaceutical formulation; the solubilizer/wetting agent component comprises from about 30% to about 40% by weight of the pharmaceutical formulation; the optional co-solubilizer component, when present, comprises from about 0.5% to about 1.5% by weight of the pharmaceutical formulation; the diluent/adsorbent component comprises from about 20% to about 26% by weight of the pharmaceutical formulation; the optional second diluent/adsorbent component, when present, comprises from about 20% to about 26% by weight of the pharmaceutical formulation; the disintegrant component comprises from about 3% to about 5% by weight of the pharmaceutical formulation; the optional glidant component, when present, comprises from about 0.1% to about 0.4% by weight of the pharmaceutical formulation; and the optional lubricant component, when present, comprises from about 0.01% to about 5% by weight of the pharmaceutical formulation.

In some embodiments, the solubilizer/wetting agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar ester of fatty acid, glyceride of fatty acid, or polyglycolized glyceride; preferably, Poloxamer 188.

In some embodiments, the co-solubilizer component comprises one or more of gelatin, polyvinylpyrrolidone (PVP), hydroxypropylmethylcellulose (HPMC), pregelatinized starch, plain starch, hydroxypropylcellulose (HPC), or carboxymethylcellulose (CMC); preferably, polyvinylpyrrolidone K17.

In some embodiments, the diluent/adsorbent component comprises one or more of carboxymethyl cellulose, carboxyethyl cellulose, hydroxyethyl cellulose, microcrystalline cellulose, starch, calcium phosphate, anhydrous dicalcium phosphate, sodium starch glycolate, metal aluminosilicate, magnesium aluminometasilicate, or metal carbonate such as magnesium carbonate; preferably, magnesium aluminometasilicate (Neusilin®).

In some embodiments, the optional second diluent/adsorbent component comprises one or more of a calcium phosphate, anhydrous dicalcium phosphate, carboxymethyl cellulose, carboxyethyl cellulose, hydroxyethyl cellulose, microcrystalline cellulose, starch, sodium starch glycolate, metal aluminosilicate, or magnesium aluminometasilicate; preferably, anhydrous dicalcium phosphate.

In some embodiments, the disintegrant component comprises one or more of croscarmellose sodium, modified cellulose, pregelatinized starch, sodium starch glycolate, crospovidone, starch, alginic acid, sodium alginate, clay, cellulose floc, ion exchange resin, silica such as Aerosil® 200, or effervescent system based on food acid and an alkaline carbonate component; preferably, croscarmellose sodium.

In some embodiments, the optional glidant component, when present, comprises one or more of starch, talc, lactose, stearate, dibasic calcium phosphate, magnesium carbonate, magnesium oxide, calcium silicate, silicon dioxide, or silicon dioxide aerogel; preferably, silicon dioxide (e.g., Syloid® 244FP).

In some embodiments, the optional lubricant component comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica such as Aerosil® 200, or sodium chloride; preferably, magnesium stearate.

In some further embodiments, the solubilizer/wetting agent component comprises Poloxamer 188; the co-solubilizer component comprises Povidone K17; the diluent/adsorbent component comprises magnesium aluminosilicate (e.g., Neusilin®); the disintegrant component comprises croscarmellose sodium; the optional second diluent/adsorbent component comprises anhydrous dicalcium phosphate; the optional glidant component, when present, comprises silicon dioxide; and the optional lubricant component comprises magnesium stearate.

In some embodiments, the formulation contains from about 1 mg to about 125 mg, or about 1 mg to about 3 mg, or about 3 mg to about 7 mg, or about 20 mg to about 30 mg, or about 40 mg to about 60 mg, or about 70 mg to about 80 mg, or about 90 mg to about 110 mg of active pharmacological agent.

The present invention further provides processes for preparing a pharmaceutical formulation comprising a pharmaceutically effective amount of an active pharmacological agent and a carrier or excipient system, the carrier or excipient system comprising:

a) a solubilizer/wetting agent component comprising from about 1% to about 60% by weight of the pharmaceutical formulation;

b) an optional co-solubilizer component comprising from about 0.04% to about 15% by weight of the pharmaceutical formulation;

c) a diluent/adsorbent component comprising from about 10% to about 60% by weight of the pharmaceutical formulation;

d) an optional second diluent/adsorbent component comprising from about 10% to about 88% by weight of the pharmaceutical formulation;

e) a disintegrant component comprising from about 0.5% to about 8% by weight of the pharmaceutical formulation;

f) an optional glidant component comprising from about 0.05% to about 5.0% by weight of the pharmaceutical formulation; and

g) an optional lubricant component comprising from about 0.001% to about 10.0% by weight of the pharmaceutical formulation; the process comprising:

i) blending the diluent/adsorbent component, at least a portion of the second diluent/adsorbent component, when present, and at least a portion of the disintegrant component to form a first mixture;

ii) mixing together the solubilizer/wetting agent component, the co-solubilizer component, if present, and the active pharmacological agent to form a second mixture;

iii) mixing the first and second mixtures to form a third mixture;

iv) blending at least a portion of the disintegrant component and the glidant component, if present, and, if present, at least a portion of the second diluent/adsorbent component with the third mixture to form a fourth mixture;

v) adding the optional lubricant component, if present, to the fourth mixture to form a final blend;

wherein the pharmacological agent is 2-(3-fluoro-4-hydroxyphenyl)-7-vinyl-1,3-benzoxazol-5-ol (ERB-041) or a pharmaceutically acceptable salt thereof.

In some embodiments, the pharmacological active agent is micronised. In some embodiments, step (i) is performed in a heated jacketed bowl.

In some embodiments, the solubilizer/wetting agent component and the co-solubilizer component are separately melted prior to mixing with the active pharmacological agent. In further embodiments, the solubilizer/wetting agent component and the co-solubilizer component are melted together prior to mixing with the active pharmacological agent, preferably at a temperature from about 110° C. to about 130° C.; preferably about 120° C.

In some embodiments, the solubilizer/wetting agent component, the co-solubilizer component and the active pharmacological agent are melted at a temperature from about 110° C. to about 130° C.; preferably 120° C.; preferably for about 30 minutes to about 4 hours; preferably until a substantially clear mixture is attained.

In some embodiments, step (iii) is performed at a temperature from about 90° C. to about 130° C.; preferably about 100° C. Typically, the heated granulate is then cooled.

In some embodiments, the processes further comprise encapsulating at least a portion of the final blend.

As will be appreciated, the formulations described herein can be prepared by a variety of procedures known for preparation of wax melt formulations. For example, in one embodiment, the dry powder can be added to a jacketed bowl and poured, pumped or sprayed in the molten polymer while mixing (with the active pharmacological agent being present in either the powder, the polymer, or both). Or, in a further embodiment, the melt can be added to the bowl (or could be melted in the bowl) and the powder can be added with mixing.

In a further embodiment, all the materials, including the wax, can be added as a powder to the jacketed bowl, and the bowl can be heated while mixing to form the melt granulation. In a still further embodiment, all the materials, including the wax, can be added as a powder to the jacketed bowl, and the impeller can be run at a high speed such that the shear and heat generated by the impeller is sufficient to melt the wax to form the melt granulation.

In a further embodiment, the melt granulation can be prepared in a fluid bed system. For example, the dry powders can be fluidized in the fluid bed bowl, and the molten wax can be sprayed on the powders. In a further embodiment, all the materials, including the wax, can be added as a powder and fluidized, increasing the air temperature to form the melt granulation. Further suitable techniques include microwave heating and extrusion/spheronization. Examples of such procedures can be found in, for example, Heng, P. W. S., and Wong, T. W., “Melt Processes for Oral Solid Dosage forms”, Encyclopedia of Pharmaceutical Technology, 1-6; Marcel Dekker, Inc., New York, 2003; Evrard, B., et al., Drug Development and Industrial Pharmacy (1999) 25(11) 1177-1184; Royce. A., et al., Drug Development and Industrial Pharmacy (1996) 22(9 &10) 917-924; Passerini, N., et al., European Journal of Pharmaceutical Sciences (2002) 15; 71-78; Gupta, M. K., et al., and Pharmaceutical Development and Technology, (2001) 6(4) 563-572, each of which is incorporated by reference herein in its entirety.

The present invention also provides products of the processes described herein.

In some embodiments, the pharmaceutical formulations of the invention comprise about 40 mg to about 60 mg ERB-041 micronised; about 90 mg to about 110 mg Poloxamer 188; about 2 mg to about 4 mg Povidone K17; about 55 mg to about 75 mg Neusilin®; about 55 mg to about 75 mg anhydrous dicalcium phosphate; about 8 mg to about 12 mg croscarmellose sodium; about 0.01 mg to about 1 mg Syloid® 244FP; and optionally, about 1.0 mg to about 2.0 mg magnesium stearate.

It will be understood that the weight percentages set forth for the diluent/adsorbent component, solubilizer/wetting agent component, optional co-solubilizer/wetting agent component, disintegrant component, optional second diluent/adsorbent component, optional glidant, and lubricant component of the formulations disclosed herein are the percentages that each component will comprise of a final pharmaceutical formulation, without reference to any surface covering, such as a tablet coating or capsule. The remainder of the final formulation will be comprised of the active pharmacological agent(s).

Generally, the active pharmacological agent(s) can be present in from about 0.5% to about 50% by weight of the pharmaceutical formulation, from about 5% to about 50% by weight of the pharmaceutical formulation, or from about 10% to about 30% by weight of the pharmaceutical formulation. In some embodiments, the active pharmacological agent comprises 2-(3-fluoro-4-hydroxyphenyl)-7-vinyl-1,3-benzoxazol-5-ol (ERB-041) or a pharmaceutically acceptable salt thereof.

The solubilizer/wetting agent generally comprises from about 1% to about 60% by weight of the pharmaceutical formulation, about 20% to about 50% by weight of the pharmaceutical formulation, or about 25% to about 35% by weight of the pharmaceutical formulation. In some embodiments, the solubilizers/wetting agent component include one or more agent that is useful as solubilizers or a wetting agent or a combination of such agents. The solubilizer/wetting agent can be any of the variety of compounds useful as solubilizing and/or wetting agents in pharmaceutical formulations, and particularly in wax melt formulations. Examples of suitable solubilizing/wetting agents include Poloxamer 188, sodium lauryl sulfate, polyoxyethylene sorbitan fatty acid esters, polyethylene glycols, polyoxyethylene castor oil derivatives, docusate sodium, quaternary ammonium amine compounds, sugar ester of fatty acids, glyceride of fatty acids, and polyglycolized glycerides. In some embodiments, the solubilizing/wetting agent is Poloxamer 188.

The optional co-solubilizing agent (co-solubilizer) is generally present in an amount of from about 0.04% to about 15% by weight of the pharmaceutical formulation, 0.1% to about 5% by weight of the pharmaceutical formulation, from about 0.5% to about 3% by weight of the pharmaceutical formulation, or about 0.5% to about 1.5% by weight of the pharmaceutical formulation. The co-solubilizer is generally selected from compounds that are useful for solubilizing pharmaceuticals in wax-melt formulations. Examples of suitable co-solubilizer include gelatin, polyvinylpyrrolidone (PVP), hydroxypropylmethylcellulose (HPMC), pregelatinized starch, plain starch, hydroxypropylcellulose (HPC), and carboxymethylcellulose (CMC). In some embodiments, the co-solubilizer is polyvinylpyrrolidone K17.

The diluent/adsorbent is generally present in an amount of about 10% to about 60% by weight of the pharmaceutical formulation, about 15% to about 30% by weight of the pharmaceutical formulation, about 18% to about 27% by weight of the pharmaceutical formulation, or about 20 to about 26% weight of the pharmaceutical formulation.

The second optional diluent/adsorbent is generally present in an amount of about 10% to about 88% by weight of the pharmaceutical formulation, about 15% to about 30% by weight of the pharmaceutical formulation, about 18% to about 27% by weight of the pharmaceutical formulation, or about 20 to about 26% weight of the pharmaceutical formulation.

Both the diluent/adsorbent and the second optional diluent/adsorbent can be any diluent and/or adsorbent compounds (and/or filler compounds) useful for preparing pharmaceutical preparations, particularly wax melt formulations. The diluent/adsorbent component amounting to from about 10% to about 60% by weight of the pharmaceutical formulation comprises one or more compounds that may be selected from the following examples. The optional second diluent/adsorbent component, if present, amounting to from about 10% to about 88% by weight of the pharmaceutical formulation comprises one or more compounds that may be selected from the following examples. Examples of suitable diluent/adsorbents include substituted celluloses, for example, carboxymethyl cellulose, ethyl cellulose, carboxyethyl cellulose, hydroxyethyl celluloses, microcrystalline celluloses, starches, calcium phosphates such as anhydrous dicalcium phosphate, sodium starch glycolates, metal aluminosilicates such as magnesium aluminometasilicate (e.g., Neusilin®), sugar or carbohydrate containing compounds such as mannitol, lactose, sucrose, maltodextrin, sorbitol, starch and xylitol, as well as metal phosphates and carbonates, for example, magnesium carbonate. Other suitable diluent/adsorbent (or filler) materials can be found in, for example, Remington's Pharmaceutical Sciences, 17th ed., Mack Publishing Company, Easton, Pa., 1985, which is incorporated herein by reference in its entirety. In some embodiments, the diluent/adsorbent component and the optional second diluent/adsorbent component include one or more agent that is useful as a diluent or a adsorbent or a combination of such agents. Each diluent/adsorbent substance used preferably is a diluent exhibiting adsorbent properties. In some embodiments, the diluent/adsorbent is magnesium aluminometasilicate (e.g., Neusilin®), and the optional second diluent/adsorbent is anhydrous dicalcium phosphate. Alternatively the diluent/adsorbent component may be a combination of magnesium aluminometasilicate (e.g., Neusilin®) and anhydrous dicalcium phosphate and the optional second diluent/adsorbent may be a combination of magnesium aluminometasilicate (e.g., Neusilin®) and anhydrous dicalcium phosphate.

The disintegrant component is generally present in an amount of from about 0.5% to about 8% by weight of the pharmaceutical formulation, about 1% to about 6% by weight of the pharmaceutical formulation, or about 3% to about 5% by weight of the pharmaceutical formulation. The disintegrant component can be selected from disintegrants known in the art, including croscarmellose sodium, modified cellulose, pregelatinized starch, sodium starch glycolate, crospovidone, starch, alginic acid, sodium alginate, clays (e.g., veegum or xanthan gum), cellulose floc, ion exchange resins, silica (e.g., Aerosil® 200), and effervescent systems such as those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.).

The formulations of the invention can optionally contain one or more glidants. Generally, the glidant is present in an amount up to about 5% by weight of the formulation, for example, from about 0.05% to about 5.0% of the weight of the pharmaceutical formulation, about 0.1% to about 1.0% by weight of the pharmaceutical formulation, or about 0.1% to about 0.4% by weight of the pharmaceutical formulation. Suitable glidants include those useful in the art, for example starch, talc, lactose, stearates, dibasic calcium phosphate, magnesium carbonate, magnesium oxide, calcium silicate, silicon dioxide, and silicon dioxide aerogels. In some embodiments, the glidant is silicon dioxide, e.g., Syloid® 244FP.

The present formulations also can contain an optional lubricant component, generally, present in an amount up to about 10% by weight of the formulation, for example from about 0.001% to about 10.0% by weight of the pharmaceutical formulation, about 0.005% to about 9% by weight of the pharmaceutical formulation, about 0.01% to about 8% by weight of the pharmaceutical formulation, or about 0.01% to about 5% by weight of the pharmaceutical formulation. Suitable lubricants include those known to be useful in the art. Examples include metallic stearates, fatty acid esters, fatty acids, fatty alcohols, glyceryl behenate, mineral oil, paraffins, hydrogenated vegetable oils, leucine, polyethylene glycols, metallic lauryl sulfates, silica such as Aerosil® 200, and sodium chloride. In some embodiments, the lubricant, when present, is magnesium stearate.

In some embodiments, the present invention includes formulations as described above that have been diluted to afford lower dosages. Accordingly, in some embodiments, the invention provides lower dose pharmaceutical formulations comprising: i) a pharmaceutical formulation as described above, comprising up to about 5% of the weight of the lower dose pharmaceutical formulation, preferably from about 3% to about 5% of the weight of the lower dose pharmaceutical formulation;

ii) a diluent/adsorbent component comprising up to about 95% by weight of the lower dose pharmaceutical formulation, preferably from about 90% to about 95% by weight of the lower dose pharmaceutical formulation;

iii) an optional disintegrant component comprising up to about 5% by weight of the lower dose pharmaceutical formulation, preferably from about 3% to about 4% of the weight of the lower dose pharmaceutical formulation;

vi) an optional glidant component comprising up to about 0.5% by weight of the lower dose pharmaceutical formulation, preferably from about 0.2% to about 0.5% by weight of the lower dose pharmaceutical formulation; and

v) an optional lubricant component comprising up to about 1.0% by weight of the lower dose pharmaceutical formulation, preferably from about 0.01% to about 0.4% by weight of the lower dose pharmaceutical formulation.

The oral formulations described herein can comprise any conventionally used oral forms, including tablets, capsules, buccal forms, troches, lozenges and oral liquids, suspensions, and the like. Capsules are preferred. Capsules or tablets containing the present formulation can also be combined with mixtures of other active compounds or inert fillers and/or diluents such as the pharmaceutically acceptable starches (e.g., corn, potato or tapioca starch), sugars, artificial sweetening agents, powdered celluloses such as crystalline and microcrystalline celluloses, flours, gelatins, gums, etc. In some embodiments, the formulations are contained in capsules, preferably made by the melt process.

Tablet formulations can be made by conventional compression methods and utilize pharmaceutically acceptable diluents/adsorbents (fillers), binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations used herein may utilize standard delay or time release formulations or spansules. Suppository formulations may be made from traditional materials, including cocoa butter, with or without the addition of waxes to alter the suppositories melting point, and glycerin. Water soluble suppository bases such as polyethylene glycols of various molecular weights may also be used.

Film coatings useful with the present formulations are known in the art and generally consist of a polymer (usually a cellulosic type of polymer), a colorant and a plasticizer. Additional ingredients such as wetting agents, sugars, flavors, oils and lubricants can be included in film coating formulations to impart certain characteristics to the film coat. The formulations and formulations herein may also be combined and processed as a solid, then placed in a capsule form such as a gelatin capsule.

As will be appreciated, some components of the formulations of the invention can possess multiple functions. For example, a given component can act as both a diluent/adsorbent and as a disintegrant. In some such cases, the function of a given component can be considered singular even though its properties may allow multiple functionality.

The pharmaceutical formulations and excipient systems herein can also contain an antioxidant or a mixture of antioxidants such as ascorbic acid. Other antioxidants that can be used include, for example, sodium ascorbate and ascorbyl palmitate, optionally in conjunction with an amount of ascorbic acid. An example range for the antioxidant(s) is from about up to about 15% by weight, e.g., from about 0.05% to about 15% by weight, from about 0.5% to about 15% by weight, or from about 0.5% to about 5% by weight. In some embodiments, the pharmaceutical formulations contain substantially no antioxidant.

Additional numerous various excipients, dosage forms, dispersing agents and the like that are suitable for use in connection with the solid dispersions of the invention are known in the art and described in, for example, Remington's Pharmaceutical Sciences, 17th ed., Mack Publishing Company, Easton, Pa., 1985, which is incorporated herein by reference in its entirety.

The materials, methods, and examples presented herein are intended to be illustrative, and are not intended to limit the scope of the invention. All publications, patent applications, patents, and other references, including books, mentioned herein are incorporated by reference in their entirety.

### EXAMPLES

### Example 1

### Procedure for Preparation of Capsules Containing ERB-041.

Amounts of components are shown in the Table below.

1. 8.516 g Neusilin®, 8.516 g Dicalcium Phosphate and 0.675 g Croscarmellose Sodium are weighed and mixed together.
2. 14.999 g Poloxamer 188 are weighed and melted in a suitable container in an oil bath at 120° C.
3. 0.45 g PVP are weighed and added to the hot melt of Step 2, and mixed until dissolved.
4. 7.5 g ERB-041 are weighed and slowly added to the melted Poloxamer while stirring vigorously.
5. The mixture from Step 1 is slowly added to the mixture from Step 4 while stirring vigorously until a uniform granulation is formed. (Granulation Temperature approx. 100° C.)
6. The granulation is cooled for 10 minutes, then passed through a mesh #30 screen.
7. The remaining 1.488 g Neusilin®, 1.488 g Dicalcium Phosphate, 0.064 g Syloid®, and 1.054 g Croscarmellose Sodium are weighed and mixed with the granulation of Step 6.

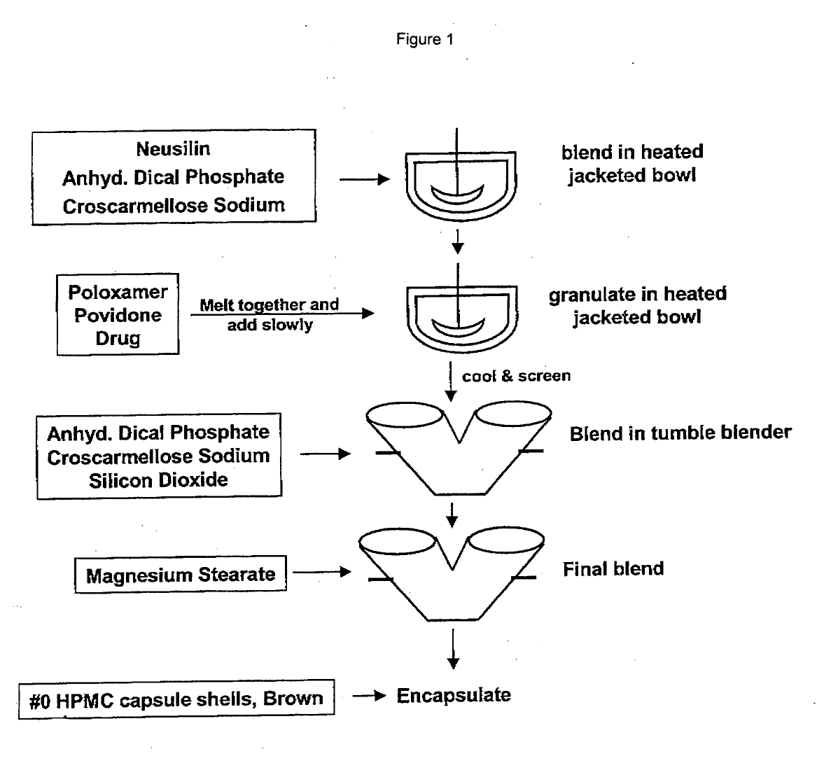

The formulation of the capsules is shown in the Table below. A flow diagram of the process is shown in FIG. 1.

In the Example above, the capsules were filled by hand, so a lubricant was not needed. However, for machine filling, it would be beneficial to include a lubricant in the formulation, as described above.

| Ingredient | % WT/WT | mg/capsule | |
|---|---|---|---|
| ERB-041 Micronised | 16.670 | 50.010 | mg |
| Poloxamer 188 NF | 33.33 | 99.990 | mg |
| Povidone K17 | 1.000 | 3.000 | mg |
| Neusilin ® | 22.425 | 67.275 | mg |
| Dicalcium Phosphate (anhydrous) | 22.425 | 67.275 | mg |
| Croscarmellose Sodium EP/NF | 4.000 | 12.000 | mg |
| Syloid ® 244FP | 0.150 | 0.450 | mg |
| TOTAL | 100.00 | 1000.00 | mg |

The present invention also includes products of the processes described herein.

As those skilled in the art will appreciate, numerous changes and modifications may be made to the preferred embodiments of the invention without departing from the spirit of the invention. It is intended that all such variations fall within the scope of the invention.

What is claimed is:

1. A pharmaceutical formulation comprising a pharmaceutically effective amount of an active pharmacological agent and a carrier or excipient system, the carrier or excipient system comprising:
a) a solubilizer/wetting agent component comprising from about 1% to about 60% by weight of the pharmaceutical formulation;
b) an optional co-solubilizer component comprising from about 0.04% to about 15% by weight of the pharmaceutical formulation;
c) a diluent/adsorbent component comprising from about 10% to about 60% by weight of the pharmaceutical formulation;
d) an optional second diluent/adsorbent component comprising from about 10% to about 88% by weight of the pharmaceutical formulation;
e) a disintegrant component comprising from about 0.5% to about 8% by weight of the pharmaceutical formulation;
f) an optional glidant component comprising from about 0.05% to about 5.0% by weight of the pharmaceutical formulation; and
g) an optional lubricant component comprising from about 0.001% to about 10.0% by weight of the pharmaceutical formulation;
wherein the active pharmacological agent has the Formula I: 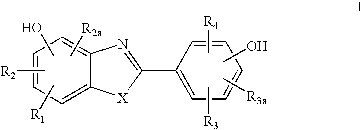
wherein
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ and R₆ are each, independently hydrogen, alkyl of 1-6 carbon atoms, or aryl of 6-10 carbon atoms;
X is O, S, or N R₇; and
R₇ is hydrogen, alkyl of 1-6 carbon atoms or aryl of 6-10 carbon atoms, —COR₅, —CO₂R₅ or —SO₂R₅;
or a pharmaceutically acceptable salt thereof.

2. The pharmaceutical formulation of claim 1 wherein X is O.

3. The pharmaceutical formulation of claim 2, wherein R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆.

4. The pharmaceutical formulation of claim 1 wherein the active ingredient is 2-(3-fluoro-4-hydroxyphenyl)-7-vinyl-1,3-benzoxazol-5-ol or a pharmaceutically acceptable salt thereof.

5. The pharmaceutical formulation of claim 1 or claim 4 wherein the active pharmacological agent comprises up to about 59% by weight of the pharmaceutical formulation.

6. The pharmaceutical formulation of claim 1 or claim 4 wherein:
the active pharmacological agent comprises from about 0.5% to about 50% by weight of the pharmaceutical formulation;
the solubilizer/wetting agent component comprises from about 20% to about 50% by weight of the pharmaceutical formulation;
the optional co-solubilizer component, when present, comprises from about 0.1% to about 5% by weight of the pharmaceutical formulation;
the diluent/adsorbent component comprises from about 15% to about 30% by weight of the pharmaceutical formulation;
the optional second diluent/adsorbent component, when present, comprises from about 15% to about 30% by weight of the pharmaceutical formulation;
the disintegrant component comprises from about 1% to about 6% by weight of the pharmaceutical formulation; and
the optional glidant component, when present, comprises from about 0.1% to about 1.0% by weight of the pharmaceutical formulation; and
the optional lubricant component, when present, comprises from about 0.005% to about 9% by weight of the pharmaceutical formulation.

7. The pharmaceutical formulation of claim 1 or claim 4 wherein:
the active pharmacological agent comprises from about 5.0% to about 50% by weight of the pharmaceutical formulation;
the solubilizer/wetting agent component comprises from about 25% to about 35% by weight of the pharmaceutical formulation;
the optional co-solubilizer component, when present, comprises from about 0.5% to about 3% by weight of the pharmaceutical formulation;
the diluent/adsorbent component comprises from about 18% to about 27% by weight of the pharmaceutical formulation;
the optional second diluent/adsorbent component, when present, comprises from about 18% to about 27% by weight of the pharmaceutical formulation;
the disintegrant component comprises from about 3% to about 5% by weight of the pharmaceutical formulation; and
the optional glidant component, when present, comprises from about 0.1% to about 0.4% by weight of the pharmaceutical formulation; and
the optional lubricant component, when present, comprises from about 0.01% to about 8% by weight of the pharmaceutical formulation.

8. The pharmaceutical formulation of claim 1 or claim 4 wherein:
the active pharmacological agent comprises from about 10% to about 30% by weight of the pharmaceutical formulation;
the solubilizer/wetting agent component comprises from about 30% to about 40% by weight of the pharmaceutical formulation;
the optional co-solubilizer component, when present, comprises from about 0.5% to about 1.5% by weight of the pharmaceutical formulation;
the diluent/adsorbent component comprises from about 20% to about 26% by weight of the pharmaceutical formulation;
the optional second diluent/adsorbent component, when present, comprises from about 20% to about 26% by weight of the pharmaceutical formulation;
the disintegrant component comprises from about 3% to about 5% by weight of the pharmaceutical formulation;
the optional glidant component, when present, comprises from about 0.1% to about 0.4% by weight of the pharmaceutical formulation; and
the optional lubricant component, when present, comprises from about 0.01% to about 5% by weight of the pharmaceutical formulation.

9. The pharmaceutical formulation of claim 1 or claim 4 wherein the solubilizer/wetting agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar ester of fatty acid, glyceride of fatty acid, or polyglycolized glyceride.

10. The pharmaceutical formulation of claim 1 or claim 4 wherein the solubilizer/wetting agent component comprises Poloxamer 188.

11. The pharmaceutical formulation of claim 1 or claim 4 wherein the optional co-solubilizer component, if present, comprises one or more of gelatin, polyvinylpyrrolidone (PVP), hydroxypropylmethylcellulose (HPMC), pregelatinized starch, plain starch, hydroxypropylcellulose (HPC), or carboxymethylcellulose (CMC).

12. The pharmaceutical formulation of claim 1 or claim 4 wherein the optional co-solubilizer component, if present, comprises polyvinylpyrrolidone K17.

13. The pharmaceutical formulation of claim 1 or claim 4 wherein the diluent/adsorbent component comprises one or more of carboxy methylcellulose, carboxyethyl cellulose, hydroxyethyl cellulose, microcrystalline cellulose, starch, calcium phosphate, anhydrous dicalcium phosphate, sodium starch glycolate, magnesium carbonate, metal aluminosilicate, or magnesium aluminometasilicate.

14. The pharmaceutical formulation of claim 1 or claim 4 wherein the diluent/adsorbent component comprises magnesium aluminometasilicate.

15. The pharmaceutical formulation of claim 1 or claim 4 wherein the optional second diluent/adsorbent component, if present, comprises one or more of a calcium phosphate, anhydrous dicalcium phosphate, carboxymethyl cellulose, carboxyethyl cellulose, hydroxyethyl cellulose, microcrystalline cellulose, starch, sodium starch glycolate, metal aluminosilicate, or magnesium aluminometasilicate.

16. The pharmaceutical formulation of claim 1 or claim 4 wherein the optional second diluent/adsorbent component, if present, comprises anhydrous dicalcium phosphate.

17. The pharmaceutical formulation of claim 1 or claim 4 wherein the disintegrant component comprises one or more of croscarmellose sodium, modified cellulose, pregelatinized starch, sodium starch glycolate, crospovidone, starch, alginic acid, sodium alginate, clay, cellulose floc, ion exchange resin, silica, or effervescent system based on food acids and an alkaline carbonate component.

18. The pharmaceutical formulation of claim 1 or claim 4 wherein the disintegrant is croscarmellose sodium.

19. The pharmaceutical formulation of claim 1 or claim 4 wherein the optional glidant component, if present, comprises one or more of starch, talc, lactose, stearate, dibasic calcium phosphate, magnesium carbonate, magnesium oxide, calcium silicate, silicon dioxide, or silicon dioxide aerogel.

20. The pharmaceutical formulation of claim 1 or claim 4 wherein the optional glidant component, if present, is silicon dioxide.

21. The pharmaceutical formulation of claim 1 or claim 4 wherein the optional lubricant component, if present, comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica, or sodium chloride.

22. The pharmaceutical formulation of claim 1 or claim 4 wherein the optional lubricant, if present, component is magnesium stearate.

23. The pharmaceutical formulation of claim 1 or claim 4 wherein:
the solubilizer/wetting agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar ester of fatty acid, glyceride of fatty acid, or polyglycolized glyceride;
the co-solubilizer component, when present, comprises one or more of gelatin, polyvinylpyrrolidone (PVP), hydroxypropylmethylcellulose (HPMC), pregelatinized starch, plain starch, hydroxypropylcellulose (HPC), or carboxymethylcellulose (CMC);
the diluent/adsorbent component comprises one or more of carboxymethyl cellulose, carboxyethyl cellulose, hydroxyethyl cellulose, microcrystalline cellulose, starch, calcium phosphate, anhydrous dicalcium phosphate, sodium starch glycolate, magnesium carbonate, metal aluminosilicate, or magnesium aluminometasilicate;
the disintegrant component comprises one or more of croscarmellose sodium, modified cellulose, pregelatinized starch, sodium starch glycolate, crospovidone, starch, alginic acid, sodium alginate, clay, cellulose floc, ion exchange resin, silica, or effervescent system based on food acid and an alkaline carbonate component;
the optional second diluent/adsorbent component, when present, comprises one or more of calcium phosphate, anhydrous dicalcium phosphate, carboxymethyl cellulose, carboxyethyl cellulose, hydroxyethyl cellulose, microcrystalline cellulose, starch, sodium starch glycolate, metal aluminosilicate, or magnesium aluminometasilicate;
the optional glidant component, when present, comprises one or more of starch, talc, lactose, stearate, dibasic calcium phosphate, magnesium carbonate, magnesium oxide, calcium silicate, silicon dioxide, or silicon dioxide aerogel; and
the optional lubricant component, when present, comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica, and sodium chloride.

24. The pharmaceutical formulation of claim 1 or claim 4 wherein:
the solubilizer/wetting agent component comprises Poloxamer 188;
the optional co-solubilizer component, when present, comprises polyvinylpyrrolidone K17;
the diluent/adsorbent component comprises magnesium aluminometasilicate;
the disintegrant component comprises croscarmellose sodium;
the optional second diluent/adsorbent component, when present, comprises anhydrous dicalcium phosphate;
the optional glidant component, when present, comprises silicon dioxide; and
the optional lubricant component, when present, comprises magnesium stearate.

25. The pharmaceutical formulation of claim 1 wherein the formulation contains from about 1 mg to about 125 mg of active pharmacological agent.

26. The pharmaceutical formulation of claim 1 wherein the formulation contains from about 1 mg to about 3 mg of active pharmacological agent.

27. The pharmaceutical formulation of claim 1 wherein the formulation contains from about 3 mg to about 7 mg of active pharmacological agent.

28. The pharmaceutical formulation of claim 1 wherein the formulation contains from about 20 mg to about 30 mg of active pharmacological agent.

29. The pharmaceutical formulation of claim 1 wherein the formulation contains from about 40 mg to about 60 mg of active pharmacological agent.

30. The pharmaceutical formulation of claim 1 wherein the formulation contains from about 70 mg to about 80 mg of active pharmacological agent.

31. The pharmaceutical formulation of claim 1 wherein the formulation contains from about 90 mg to about 110 mg of active pharmacological agent.

32. The pharmaceutical formulation of claim 5 wherein the formulation contains from about 1 mg to about 125 mg of active pharmacological agent.

33. The pharmaceutical formulation of claim 5 wherein the formulation contains from about 1 mg to about 3 mg of active pharmacological agent.

34. The pharmaceutical formulation of claim 5 wherein the formulation contains from about 3 mg to about 7 mg of active pharmacological agent.

35. The pharmaceutical formulation of claim 5 wherein the formulation contains from about 20 mg to about 30 mg of active pharmacological agent.

36. The pharmaceutical formulation of claim 5 wherein the formulation contains from about 40 mg to about 60 mg of active pharmacological agent.

37. The pharmaceutical formulation of claim 5 wherein the formulation contains from about 70 mg to about 80 mg of active pharmacological agent.

38. The pharmaceutical formulation of claim 5 wherein the formulation contains from about 90 mg to about 110 mg of active pharmacological agent.

39. A process for preparing a pharmaceutical formulation comprising a pharmaceutically effective amount of an active pharmacological agent and a carrier or excipient system, the carrier or excipient system comprising:
a) a solubilizer/wetting agent component comprising from about 1% to about 60% by weight of the pharmaceutical formulation;
b) an optional co-solubilizer component comprising from about 0.04% to about 15% by weight of the pharmaceutical formulation;
c) a diluent/adsorbent component comprising from about 10% to about 60% by weight of the pharmaceutical formulation;
d) an optional second diluent/adsorbent component comprising from about 10% to about 88% by weight of the pharmaceutical formulation;
e) a disintegrant component comprising from about 0.5% to about 8% by weight of the pharmaceutical formulation;
f) an optional glidant component comprising from about 0.05% to about 5.0% by weight of the pharmaceutical formulation; and
g) an optional lubricant component comprising from about 0.001% to about 10.0% by weight of the pharmaceutical formulation;
the process comprising:
i) blending the diluent/adsorbent component, at least a portion of the second diluent/adsorbent component, when present, and at least a portion of the disintegrant component to form a first mixture;
ii) mixing together the solubilizer/wetting agent component, the co-solubilizer component, when present, and the active pharmacological agent to form a second mixture;
iii) mixing the first and second mixtures to form a third mixture;
iv) blending at least a portion of the disintegrant component and the glidant component, if present, and, if present, at least a portion of the second diluent/adsorbent component with the third mixture to form a fourth mixture;
v) adding the optional lubricant component, if present, to the fourth mixture to form a final blend;
wherein the pharmacological agent is 2-(3-fluoro-4-hydroxyphenyl)-7-vinyl-1,3-benzoxazol-5-ol or a pharmaceutically acceptable salt thereof.

40. The process of claim 39 wherein the pharmacological active agent is micronised.

41. The process of claim 39 wherein:
the solubilizer/wetting agent component comprises one or more of Poloxamer 188, sodium lauryl sulfate, polyoxyethylene sorbitan fatty acid ester, polyethylene glycol, polyoxyethylene castor oil derivative, docusate sodium, quaternary ammonium amine compound, sugar ester of fatty acid, glyceride of fatty acid, or polyglycolized glyceride;
the co-solubilizer component, when present, comprises one or more of gelatin, polyvinylpyrrolidone (PVP), hydroxypropylmethylcellulose (HPMC), pregelatinized starch, plain starch, hydroxypropylcellulose (HPC), or carboxymethylcellulose (CMC);
the diluent/adsorbent component comprises one or more of carboxymethyl cellulose, carboxyethyl cellulose, hydroxyethyl cellulose, microcrystalline cellulose, starch, calcium phosphate, anhydrous dicalcium phosphate, sodium starch glycolate, magnesium carbonate, metal aluminosilicate, or magnesium aluminometasilicate;
the disintegrant component comprises one or more of croscarmellose sodium, modified cellulose, pregelatinized starch, sodium starch glycolate, crospovidone, starch, alginic acid, sodium alginate, clay, cellulose floc, ion exchange resin, silica, or effervescent system based on food acids and an alkaline carbonate component;
the optional second diluent/adsorbent component, when present, comprises one or more of a calcium phosphate, anhydrous dicalcium phosphate, carboxymethyl cellulose, carboxyethyl cellulose, hydroxyethyl cellulose, microcrystalline cellulose, starch, sodium starch glycolate, metal aluminosilicate, or magnesium aluminometasilicate;
the optional glidant component, when present, comprises one or more of starch, talc, lactose, stearate, dibasic calcium phosphate, magnesium carbonate, magnesium oxide, calcium silicate, silicon dioxide, or silicon dioxide aerogel; and
the optional lubricant component, when present, comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica, or sodium chloride.

42. The process of claim 39 wherein:
the solubilizer/wetting agent component comprises Poloxamer 188;
the co-solubilizer component, when present, comprises polyvinylpyrrolidone K17;
the diluent/adsorbent component comprises magnesium aluminometasilicate;
the disintegrant component comprises croscarmellose sodium;
the optional second diluent/adsorbent component, when present, comprises anhydrous dicalcium phosphate;
the glidant component, when present, comprises silicon dioxide; and
the optional lubricant component, when present, comprises magnesium stearate.

43. The process of claim 39 wherein (i) is performed in a heated jacketed bowl.

44. The process of claim 39 wherein the solubilizer/wetting agent component and the co-solubilizer component are separately melted prior to mixing with the active pharmacological agent.

45. The process of claim 39 wherein the solubilizer/wetting agent component and the co-solubilizer component are melted together prior to mixing with the active pharmacological agent.

46. The process of claim 44 or 45 wherein the solubilizer/wetting agent component and the co-solubilizer component are melted at a temperature from about 110° C. to about 130° C.

47. The process of claim 44 or 45 wherein the solubilizer/wetting agent component and the co-solubilizer component are melted at a temperature of about 120° C.

48. The process of claim 39 wherein the solubilizer/wetting agent component, the co-solubilizer component and the active pharmacological are melted at a temperature from about 110° C. to about 130° C.

49. The process of claim 39 wherein the solubilizer/wetting agent component, the co-solubilizer component and the active pharmacological are melted at a temperature of about 120° C.

50. The process of claim 39 wherein the solubilizer/wetting agent component, the co-solubilizer component and the active pharmacological are melted for about 30 minutes to about 4 hours.

51. The process of claim 39 wherein the solubilizer/wetting agent component, the co-solubilizer component and the active pharmacological are melted until a substantially clear mixture is attained.

52. The process of claim 39 wherein (iii) is performed at a temperature from about 90° C. to about 130° C.

53. The process of claim 39 wherein (iii) is performed at a temperature of about 100° C.

54. The process of claim 39 further comprising encapsulating at least a portion of the final blend.

55. The process of claim 39 wherein the formulation contains from about 1 mg to about 125 mg of active pharmacological agent.

56. The process of claim 39 wherein the formulation contains from about 1 mg to about 3 mg of active pharmacological agent.

57. The process of claim 39 wherein the formulation contains from about 3 mg to about 7 mg of active pharmacological agent.

58. The process of claim 39 wherein the formulation contains from about 20 mg to about 30 mg of active pharmacological agent.

59. The process of claim 39 wherein the formulation contains from about 70 mg to about 80 mg of active pharmacological agent.

60. The process of claim 39 wherein the formulation contains from about 90 mg to about 110 mg of active pharmacological agent.

61. A product of the process of any of claims 39-45 and 48-60.

62. A product of the process of claim 46.

63. A product of the process of claim 47.

64. The pharmaceutical formulation of claim 1 or claim 4 wherein the formulation comprises:
(a) about 40 mg to about 60 mg ERB-041 micronised;
(b) about 90 mg to about 110 mg Poloxamer 188;
(c) about 2 mg to about 4 mg polyvinylpyrrolidone K17;
(d) about 55 mg to about 75 mg magnesium aluminometasilicate;
(e) about 55 mg to about 75 mg anhydrous dicalcium phosphate;
(f) about 8 mg to about 12 mg croscarmellose sodium;
(g) about 0.01 mg to about 1 mg silicon dioxide; and
(h) about 1.0 mg to about 2.0 mg magnesium stearate.

65. A pharmaceutical formulation comprising:
a) a formulation according to claim 1 comprising up to about 5% of the weight of the pharmaceutical formulation; and
b) a diluent/adsorbent component comprising up to about 95% by weight of the pharmaceutical formulation;
c) an optional disintegrant component comprising up to about 5% by weight of the pharmaceutical formulation;
e) an optional glidant component comprising up to about 0.5% by weight of the pharmaceutical formulation; and
f) an optional lubricant component comprising up to about 1.0% by weight of the pharmaceutical formulation.

66. The pharmaceutical formulation of claim 65 wherein:
a) the formulation according to claim 1 comprises from about 3% to about 5% of the weight of the pharmaceutical formulation; and
b) the optional diluent/adsorbent component comprises from about 90% to about 95% by weight of the pharmaceutical formulation;
c) the optional disintegrant component, when present, comprises from about 3% to about 4% of the weight of the pharmaceutical formulation; and
e) an optional glidant component, when present, comprises from about 0.2% to about 0.5% by weight of the pharmaceutical formulation; and
f) the optional lubricant component, when present, comprises from about 0.01% to about 0.4% by weight of the pharmaceutical formulation.

67. A capsule or tablet comprising a pharmaceutical formulation of claim 1.
